(19) United States
(12) Patent Application Publication
(10) Pub. No.: US 2010/0292472 A1
(43) Pub. Date: Nov. 18, 2010

(54) PROPHYLACTIC AND THERAPEUTIC MEDICINE FOR MALARIA

(76) Inventors: Tomoko Toyama, Osaka (JP); Kisaburo Nagamune, Tokyo (JP); Toshihiro Horii, Osaka (JP); Kazuyuki Tanabe, Osaka (JP)

Correspondence Address: Kubotera & Associates, LLC, 200 Daingerfield Rd, Suite 202, Alexandria, VA, 22314, US

(21) Appl. No.: 12/863,810

(22) PCT Filed: Jan. 22, 2009
(86) PCT No.: PCT/JP2009/050925

§ 371 Date: Jul. 21, 2010

(30) Foreign Application Priority Data
Jan. 23, 2008 (JP) 2008-012693

(51) Int. Cl. C07D 473/34; C07D 285/06; C07D 213/50; C07D 249/08; C07D 295/192

(52) U.S. Cl. 544/277; 548/127; 546/340; 548/267.8; 548/268.4; 546/226

(57) ABSTRACT

The present invention can provide a novel medicament that is safe, cheap and effective for prophylaxis or therapy of apicomplexan infections, such as malaria and toxoplasmosis by applying a plant growth regulator selected from the group consisting of gibberellin inhibitors, cytokinins and ethylene inhibitors to the prophylactic or therapeutic treatment of such apicomplexan infections.

### TECHNICAL FIELD

The invention relates to a prophylactic and therapeutic medicine effective for infectious diseases caused by apicomplexan parasites, such as malaria, toxoplasmosis, cryptosporidiosis, coccidiosis and babesiosis. In particular, the present invention relates to a prophylactic and therapeutic medicine effective for human malaria caused by Plasmodia or other apicomplexan infections in mammals including livestock such as cattle and swine.

### BACKGROUND ART

Malaria is one of the three major infectious diseases, reportedly causing about 500 million infections and more than one million deaths per year in the world, notably in the tropics. Pathogenic organisms Plasmodia belong to eukaryotes and develop resistance to antimalarial medicines for a short period. This situation makes development of curative drugs quite difficult. At present, only a few kinds of therapeutic medicines for malaria are available and most of them are expensive. Most of malaria patients are in developing countries, and under such a circumstance, development of cheaper and more effective drugs is strongly demanded.

Human malaria is caused by four kinds of pathogens (Plasmodium falciparum, P. vivax, P. malariae and P. ovale) belonging to the phylum Apicomplexa. Infection to human is achieved by blood sucking by infected mosquito species of the genus Anopheles. Malaria is a severe epidemic disease in many developing countries in tropics. Fortunately, malaria infection has been very rare in Japan, but about 100 tourists are annually reported to have got infected in tropical regions and developed malaria after returning home.

Control of malaria is no easy task because of the continuous emergence of drug resistance to current antimalarial medicines and no preventive vaccines. Controlling malaria has been extensively discussed in annual summit conferences of industrially advanced nations, and Japan has been also urged to devote itself to various international frameworks.

Quinine, chloroquine, mefloquine, fansidar (sulfadoxine), primaquine and artemisinin (derived from sweet wormwood belonging to the genus Artemisia) have been well known as antimalarial medicines. However, quinine shows side effects, such as optic nerve, hematological and cardiac disorders. To substitute quinine, chloroquine and mefloquine were developed, but chloroquine is a risky drug because it causes serious adverse effects, such as chloroquine retinopathy, and is mutagenic and teratogenic. Even mefloquine, which reportedly causes dizziness, mental confusion and other undesirable effects, is regarded as a difficult drug to prescribe.

One example of new compounds invented to use for the prophylactic or therapeutic treatment of malaria is phenazines, especially riminophenazines, which have a substituted imino group in one benzene ring. In particular, N,5-bis-(phenyl)-3,5-dihydro-3-(cyclohexylimino)-2-phenazinamine has been reported to show antimalarial activity (Patent literature 1).

Besides, it has been in the public domain that a vaccine composition containing various antigens derived from malaria parasites and an adjuvant predominantly stimulating Th1 immune cells is used for prophylaxis of malaria (Patent literature 2). Febrifugine, isofebrifugine and their derivatives synthesized from chiral 3-piperidinol are shown to have a high activity against Plasmodium spp. (Non-patent literature 1). The inventors have reported that fluridone, an abscisic acid (ABA) biosynthetic inhibitor (ABA is one of plant hormones), inhibits parasite egress from infected host cells in the apicomplexan parasite Toxoplasma gondii (Non-patent literature 2).
[Patent literature 1] Japanese Patent Publication No. 08-231401
[Patent literature 2] International Patent Publication No. WO 98/05335
[Non-patent literature 1] Y. Takaya et al., New type of febrifugine analogues, bearing a quinolizidine moiety, show potent antimalarial activity against Plasmodium malaria parasite. J. Med. Chem. 42, 3163-3166 (1999)
[Non-patent literature 2] Kisaburo Nagamune et al., Abscisic acid controls calcium-dependent egress and development in Toxoplasma gondii, Nature 451, 207-210 (2008)

### DISCLOSURE OF THE INVENTION

### Problem to be Solved by the Invention

An object of the invention is to provide a novel medicine effective for prophylaxis or therapy of malaria through finding a novel effect of known safe and inexpensive compounds.

### Means for Solving the Problem

The invention includes the following aspects to solve the abovementioned problems.

[1] A prophylactic and therapeutic medicine for an apicomplexan infection, comprising a plant growth regulator selected from the group consisting of a gibberellin inhibitor, a cytokinin and an ethylene inhibitor
[2] The prophylactic and therapeutic medicine according to [1], wherein the gibberellin inhibitor is at least one selected from the group consisting of inabenfide, paclobutrazol, uniconazole P, AMO-1618 and FC-907
[3] The prophylactic and therapeutic medicine according to [1], wherein the cytokinin is at least one selected from the group consisting of thidiazuron, 6-benzyl aminopurine, trans-zeatin and cis-zeatin
[4] The prophylactic and therapeutic medicine according to [1], wherein the ethylene inhibitor is α-aminooxy acetic acid
[5] The prophylactic and therapeutic medicine according to one of [1] to [4], wherein the apicomplexan infection is malaria or toxoplasmosis
[6] Use of a gibberellin inhibitor, a cytokinin or an ethylene inhibitor for production of prophylactic and therapeutic medicines for apicomplexan infections
[7] A prophylactic or therapeutic method for apicomplexan infections, characterized by use of a gibberellin inhibitor, a cytokinin or an ethylene inhibitor

### EFFECTS OF THE INVENTION

According to the present invention, the prophylactic and therapeutic medicine for apicomplexan infections is effective for toxoplasmosis, cryptosporidiosis, coccidiosis, babesiosis and other apicomplexan infections, as well as malaria caused by Plasmodia. The medicine is also effective for chloroquine-resistant FCR-3 and CDC1 strains, as well as a drug-sensitive 3D7 strain of tropical malaria parasite Plasmodium falciparum.

According to the present invention, the compounds contained in the prophylactic and therapeutic medicine for apicomplexan infections as an active ingredient have been commonly used in agriculture for years and can be produced in bulk very easily and inexpensively by well-established manufacturing methods and facilities. In addition, because the compounds have been used for years, accumulated data on their toxicity and teratogenicity are available, and most advantageously, the compounds are safe in human or other animals and useful plants.

### BRIEF DESCRIPTION OF THE DRAWINGS

FIG. 1 is a graph showing a relationship between a concentration of AMO-1618 and a growth inhibition rate of Plasmodium falciparum.

FIG. 2 is a graph showing a relationship between a concentration of inabenfide and a growth inhibition rate of P. falciparum.

FIG. 3 is a graph showing a relationship between a concentration of thidiazuron and a growth inhibition rate of P. falciparum.

FIG. 4 is a graph showing a relationship between a concentration of α-aminooxy acetic acid and a growth inhibition rate of P. falciparum.

### DETAILED DESCRIPTION OF THE PREFERRED EMBODIMENTS

During extensive investigation, the inventors incidentally found a compound inhibiting life cycles and propagation of malaria parasites among substances associated with plant growth. As a result of thorough screening of such substances, the inventors found many effective compounds for inhibiting life cycles and propagation of malaria parasites among gibberellin inhibitors, cytokinins and ethylene inhibitors.

That is, those compounds are gibberellin inhibitors, cytokinins or ethylene inhibitors known as ‘plant growth regulators’, chemicals controlling plant growth. They have been well known per se and extensively used in agriculture. The inventors found that such known compounds inhibit life cycles and propagation of apicomplexan parasites, especially of malaria parasites, conducted further investigation and finally completed the present invention. The present invention is based on such a surprising novel effect of the compounds and provides a novel antimalarial medicament by applying the above-mentioned compounds known per se to prophylaxis or therapy of apicomplexan infections such as malaria and toxoplasmosis.

Further research would be necessary to unravel accurate mechanisms in which the compounds affect physiology and morphology of parasite cells and result in growth blockage of Plasmodia. However, the following mechanisms can be considered at present. Gibberellin inhibitors might stop supply of some essential molecules for parasite survival by interrupting biosynthetic enzymes in apicoplast, an intracellular organelle related to chloroplast in Apicomplexa, resulting in termination of cell proliferation. Cytokinins regulate cell division in plants, affecting shoot formation, senescence inhibition, etc., and exogenous cytokinins might disturb effects of endogenous cytokinins synthesized in parasite cells, resulting in termination of cell proliferation. These presumptive mechanisms might also function in other apicomplexan parasites, such as Toxoplasma spp., Cryptosporidium spp., Coccidium spp. and Babesia spp.

In this description, ‘gibberellin inhibitors’ mean a group of compounds blocking effects of gibberellins, plant hormones simulating stem elongation, germination, flowering and other physiological changes. Preferable examples of the gibberellin inhibitor include inabenfide, paclobutrazol, uniconazole P, AMO-1618 and FC-907. These compounds are well known and, except for FC-907, available as commercial items. FC-907 can be synthesized according to the instruction in a literature [P. Hedden et al., Metabolism of kaurenoids by Gibberella fujikuroi in the presence of the plant growth retardant, N,N,N-trimethyl-1-methyl-(2′,6′,6′-trimethylcyclohex-2′-en-1′-yl) prop-2-enylammonium iodide. Phytochemistry 16, 1913-1917 (1977)]. Derivatives derived from the compounds are also preferable as the gibberellin inhibitor as far as they show inhibitory effects on gibberellins.

In this description, ‘cytokinins’ mean a group of compounds that promote formation of callus and shoots, axillary bud growth and the like (namely, cytokinin activity). Preferable examples of the cytokinin include thidiazuron, 6-benzyl aminopurine, trans-zeatin, cis-zeatin, isopentenyl adenine and kinetin. These compounds are well known and available as commercial items. Derivatives derived from the compounds are also preferable as the cytokinin as far as they show cytokinin activity.

In this description, ‘ethylene inhibitors’ mean a group of compounds blocking effects of ethylene, a plant hormone stimulating organ senescence, leaf abscission and fruit ripening, and affecting shoot morphology. Preferable examples of the ethylene inhibitor include compounds such as α-aminooxy acetic acid. This compound is well known and available as a commercial item. Its derivatives are also preferable as the ethylene inhibitor as far as they show inhibitory effects on ethylene.

The above-mentioned various compounds which the medicament of the present invention comprises as an active ingredient are processed into a dosage form suitable for the administration route by mixing with an appropriately selected additive (excipients, binders, disintegrants, lubricants, corrigents, solubilizers, etc.) and shaping the mixture. Examples of the dosage form include preparations suitable for oral administration (for example, tablets, capsules, granules, subtle granules, syrups, etc.), injections for subcutaneous or intravenous administration, and suppositories and nose drops for mucous administration.

Tablets, the most general dosage form among the above ones, can be prepared as follows. The compound is mixed with an excipient appropriately selected from lactose, glucose, sucrose, starch, maize starch, potato starch, rice starch, pregelatinized starch, crystalline cellulose, sorbitol, mannitol, etc., optionally with a disintegrant appropriately selected from carboxymethyl cellulose, starch, croscarmellose sodium, etc. To the mixture, a binder appropriately selected from starch paste, hydroxypropylcellulose, hydroxypropylmethylcellulose, carboxymethyl cellulose, gum arabic, gelatin, etc., is added to strengthen the binding of the ingredients to each other. The mixture is granulated by an extrusion, stirring or fluidized bed granulation method, etc. To the resulting granules is added a lubricant appropriately selected from magnesium stearate, talc, Aerosil (silica), calcium stearate, etc., and optionally a corrigent appropriately selected from menthol, sage, etc., and then the resulting mixture is compressed into tablets. Obtained tablets can be utilized with or without coating by film or sugar in accordance with common protocols.

Other dosage forms suitable for the administration route, e.g. capsules, syrups, granules, subtle granules, injections, suppositories, nose drops, or the like, can be prepared in accordance with General Rules for Preparations of the Japanese Pharmacopoeia. The suitable content of an antimalarial ingredient in such a pharmaceutical preparation should be set in such a manner that the daily dosage of the ingredient is from about 0.1 to 1500 mg/kg, and more preferably about 1 to 1000 mg/kg. It would be obvious that dosages should be changed depending on the conditions of recipients.

The present invention will be further explained by the following examples. It is to be understood, however, that the invention is not limited to the examples.

### Example 1

### Test for Growth Inhibitory Activity Against P. falciparum

To investigate growth inhibitory activity against a laboratory strain 3D7 of P. falciparum, the following various compounds were tested: inabenfide (Wako Pure Chemicals, Osaka, Japan), uniconazole P (Wako Pure Chemicals, Osaka, Japan), paclobutrazol (Wako Pure Chemicals, Osaka, Japan), AMO-1618 (CALBIOCHEM, La Jolla, Calif.) and FC-907 as gibberellin inhibitors; thidiazuron (Wako Pure Chemicals, Osaka, Japan), 6-benzyl aminopurine (Wako Pure Chemicals, Osaka, Japan), trans-zeatin (Wako Pure Chemicals, Osaka, Japan) and cis-zeatin (Sigma) as cytokinins; and α-aminooxy acetic acid (Wako Pure Chemicals, Osaka, Japan) as an ethylene inhibitor.

As a culture medium for the P. falciparum strain 3D7, filter-sterilized RPMI 1640 (pH 7.4) containing 10% (v/v) human serum and human erythrocytes at 3% hematocrit (the ratio of erythrocytes in a suspension thereof) was prepared. Each compound was dissolved at predetermined concentrations in an appropriate solvent (water, ethanol or DMSO) and added to wells. To each well, 3D7-infected erythrocytes were added at a starting parasitemia of 0.1% and the total volume was adjusted to 2.5 ml with the culture medium. The plates were incubated for 3 days. Control cultures were prepared so that instead of each compound solution, the equal volume of the corresponding solvent alone was contained, and similarly incubated. After the incubation, a thin blood film from each culture was prepared and stained with Giemsa (E. Merck, Germany). A total of 3000-erythrocytes/1 thin blood film were examined microscopically (immersed in oil, magnified to 1000 times), after which the malarial infection rate was determined using the equation below. The culture was maintained at 37° C. in a CO₂—O₂—N₂ incubator (5% CO₂, 5% O₂, and 90% N₂ atmosphere). The experiments were performed at least twice and each treatment was performed in triplicate. By comparison of the malarial infection rates obtained in control and compound-treated samples, the efficacy of each compound was assessed. The compound showing the malarial infection rate of 50% or less on average was regard as effective.

Malarial infection rate=[(number of infected erythrocytes)/(total number of examined erythrocytes)]×100

For compounds showing growth inhibitory activity, the 50% growth inhibition concentration (EC₅₀) was calculated using the following equation.

EC₅₀={(50−Y1)×(X2−X1)/(Y2−Y1)}+X1

X1: concentration of the compound showing <50% inhibition rate (μM)
Y1: inhibition rate at the concentration X1
X2: concentration of the compound showing >50% inhibition rate (μM)
Y2: inhibition rate at the concentration X2

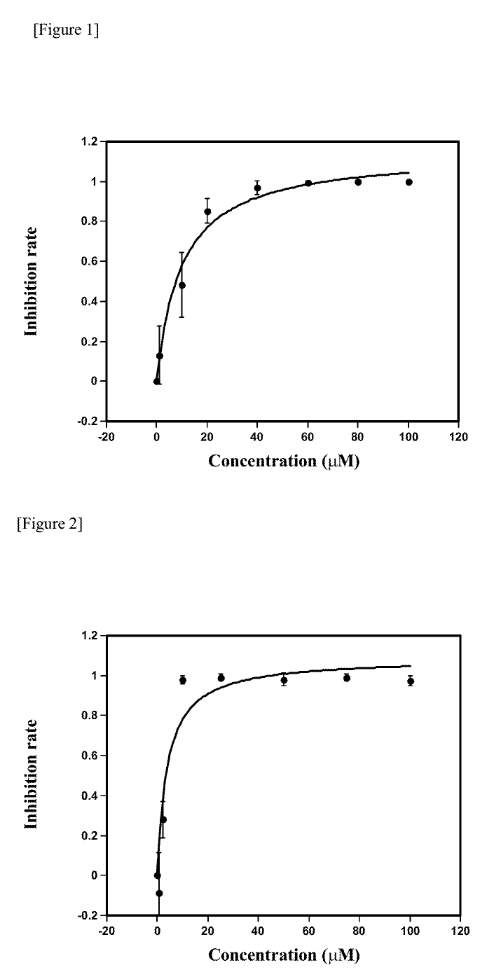

FIG. 1 shows a graph showing the relationship between the concentration of AMO-1618 (a gibberellin inhibitor) and the growth inhibition rate of P. falciparum. From this graph, values corresponding to parameters in the abovementioned equation are as follows; X1=10, Y1=48.9, X2=20, Y2=85.4. By substitution of these values for the corresponding parameters, EC₅₀ (ED₅₀) was calculated to be 10.3 μM.

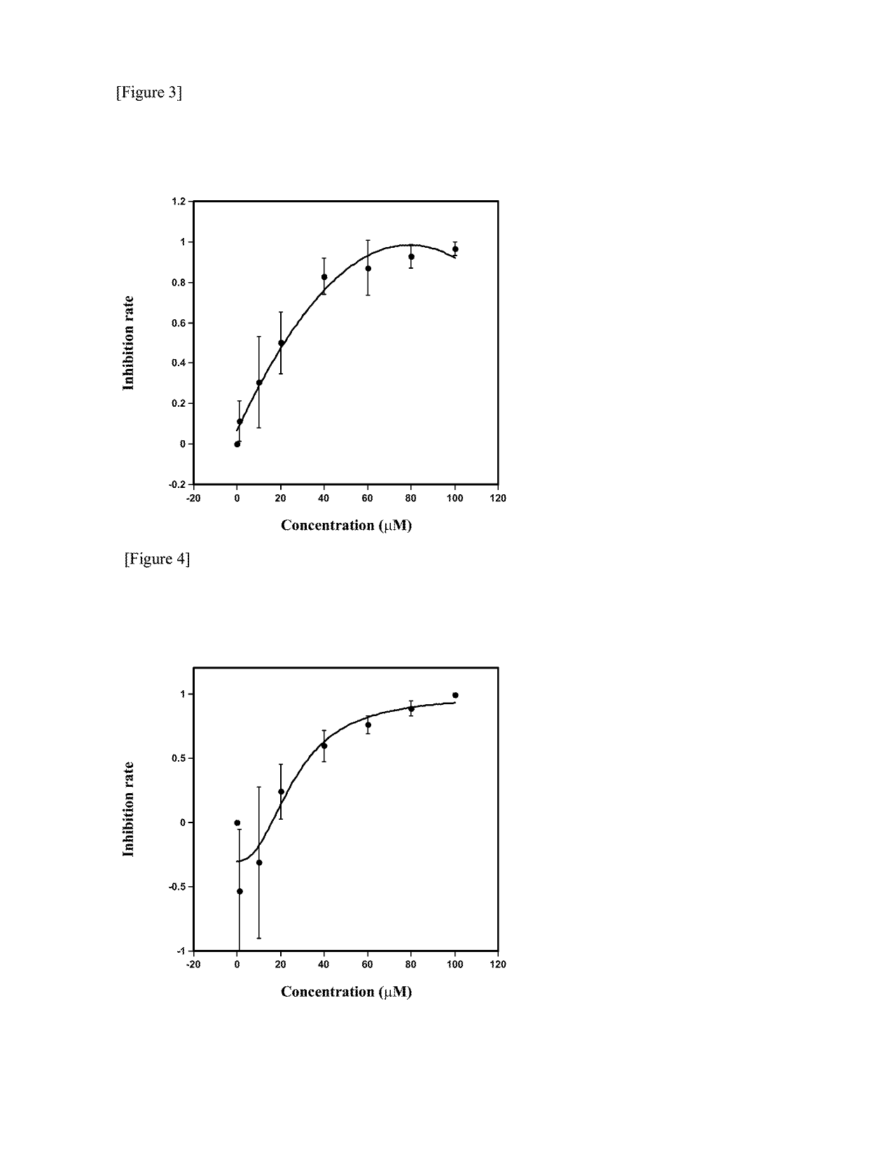

EC₅₀s were also calculated for the other compounds as mentioned above. The results are shown in Table 1. FIGS. 2 to 4 show graphs each showing the relationship between the concentration of inabenfide (a gibberellin inhibitor), thidiazuron (a cytokinin) or α-aminooxy acetic acid (an ethylene inhibitor) and the growth inhibition rate of P. falciparum.

TABLE 1

| | | | Reported Data | |
|---|---|---|---|---|
| | | | LD₅₀ | |
| Regulators | Reagents | ED₅₀ (μM) | (Mice, mg/kg) | Mutagenesis |
| Cytokinins | Thidiazuron | 17.5 | >5000 | No |
| | 6-Benzylaminopurine | 19.0 | 1300 | No |
| | trans-Zeatin | 651.4 | 1300 | — |
| | cis-Zeatin | 742.0 | — | — |
| Gibberellin | Inabenfide | 4.47 | >15000 | No |
| Inhibitors | Paclobutrazol | 27.1 | >5000 | No |
| | Uniconazole P | 25.0 | 1000 | No |
| | AMO-1618 | 10.3 | 0.36 | — |
| | FC-907 | 4.26 | — | — |
| Ethylene | α-Aminooxy acetic | 20.3 | — | — |
| Inhibitor | Acid | | | |

### Example 2

### Test for Growth Inhibitory Activity Against T. gondii

To investigate growth inhibitory activity against T. gondii, the following various compounds were tested: inabenfide (Wako Pure Chemicals, Osaka, Japan), paclobutrazol (Wako Pure Chemicals, Osaka, Japan), uniconazole P (Wako Pure Chemicals, Osaka, Japan) and AMO-1618 (CALBIOCHEM, La Jolla, Calif.) as gibberellin inhibitors; thidiazuron (Wako Pure Chemicals, Osaka, Japan) and 6-benzyl aminopurine (Wako Pure Chemicals, Osaka, Japan) as cytokinins; and α-aminooxy acetic acid (Wako Pure Chemicals, Osaka, Japan) as an ethylene inhibitor.

For this test, T. gondii strain 2F tachyzoites were used. The strain 2F was established by transfection of the T. gondii strain RH with a β-galactosidase gene (β-gal, derived from Escherichia coli) [Reference: J. M. Dobrowolski and L. D. Sibley. Toxoplasma invasion of mammalian cells is powered by the actin cytoskeleton of the parasite. Cell 84, 933-939 (1996)]. The 2F strain was kindly provided by Dr. L. David Sibley (Washington University School of Medicine, St. Louis, Mo., US). As a host cell for the parasites, Vero cells were used. Vero cell cultures were incubated with RPMI 1640 medium containing 10% (v/v) FCS in 96-well plates for 2 days and then infected with 2.5×10⁵ tachyzoites/well in RPMI 1640 medium containing 3% (v/v) FCS and various concentrations of the compounds. The tachyzoites were harvested after 2 days and the proliferation rate was monitored as a measure of β-gal activity in a colorimetric assay using chlorophenol red β-D-galactopyranoside. The proliferation rate of compound-untreated tachyzoites is set to 100% and the IC₅₀ means the concentration of the compound that inhibits the proliferation rate by 50%.

The results on T. gondii are shown in Table 2. As shown in Table 2, the compounds blocking growth of P. falciparum also inhibited proliferation of T. gondii, except for 6-benzyl aminopurine and AMO-1618. Various somatic cells in birds and mammals can be hosts of Toxoplasma, but Plasmodia can parasitize only erythrocytes in reptiles, birds and mammals. Their life cycles also distinctively differ from each other. Considering the inhibitory effects on the growth of both parasites, it has been suggested that these compounds are effective in growth inhibition of various apicomplexan parasites.

TABLE 2

| | | IC₅₀ (μM) | |
|---|---|---|---|
| Regulators | Reagents | Toxoplasma | Plasmodium |
| Cytokinins | Thidiazuron | 74.5 | 17.5 |
| | 6-Benzylaminopurine | >300 | 19.0 |
| Gibberellin | Inabenfide | 17.0 | 4.47 |
| Inhibitors | Paclobutrazol | 120.6 | 27.1 |
| | Uniconazole P | 97.4 | 25.0 |
| | AMO-1618 | >1000 | 10.3 |
| Ethylene | α-Aminooxy acetic | 96.9 | 20.3 |
| Inhibitors | Acid | | |

### Example 3

### Tablet Production

After scaling 150 g of inabenfide, 550 g of lactose and 200 g of microcrystalline cellulose, all the ingredients were placed in a fluidized bed granulator. In the granulator, 30 g of hydroxypropylcellulose as a binder was sprayed in its 5% aqueous solution. To resulting granules, 50 g of carboxymethylcellulose and 20 g of magnesium stearate were added and mixed as a disintegrant and a lubricant, respectively. The obtained mixture was compressed into tablets each weighing 100 mg.

The invention is not meant to be limited to the embodiments and examples described above. Various changes within the scope of the claims are possible, and other embodiments based on various combinations of the technical means described in different embodiments are also included within the technical scope of the present invention. All the academic publications and patent literatures cited in the description are incorporated herein by reference.

### INDUSTRIAL APPLICABILITY

The prophylactic and therapeutic medicine for apicomplexan infections according to the present invention is effective for not only malaria caused by Plasmodia, but also toxoplasmosis caused by Toxoplasma and other apicomplexan infections, and in addition, is useful as a medicine for prophylaxis or therapy of apicomplexan infections.

1. A prophylactic and therapeutic medicine for an apicomplexan infection, comprising a plant growth regulator selected from the group consisting of a gibberellin inhibitor and a cytokinin.

2. The prophylactic and therapeutic medicine according to claim 1, wherein the gibberellin inhibitor is at least one selected from the group consisting of inabenfide, paclobutrazol, uniconazole P, AMO-1618 and FC-907.

3. The prophylactic and therapeutic medicine according to claim 1, wherein the cytokinin is at least one selected from the group consisting of thidiazuron, 6-benzyl aminopurine, trans-zeatin and cis-zeatin.

4-5. (canceled)

6. A method for producing a prophylactic and therapeutic medicine for an apicomplexan infection, comprising the step of compounding a gibberellin inhibitor or a cytokinin.
